(19) United States
(12) Patent Application Publication
(10) Pub. No.: US 2025/0072716 A1
(43) Pub. Date: Mar. 6, 2025

(54) DENTAL ENDOSCOPE SYSTEM

(71) Applicant: JUN HONG JUNG, Seoul (KR)

(72) Inventor: JUN HONG JUNG, Seoul (KR)

(21) Appl. No.: 18/740,545

(22) Filed: Jun. 12, 2024

(30) Foreign Application Priority Data
Aug. 29, 2023 (KR) 10-2023-0113504

(51) Int. Cl. A61B 1/00; A61B 1/04; A61B 1/06; A61B 1/12; A61B 1/233

(52) U.S. Cl. CPC A61B 1/00032; A61B 1/00016; A61B 1/00045; A61B 1/00105; A61B 1/042; A61B 1/0669; A61B 1/121; A61B 1/233

(57) ABSTRACT

The present disclosure relates to a dental endoscope system including a movable main body unit, a power supply unit provided in the main body unit, an endoscope unit connected to the power supply unit, a monitor mount which is rotatably and foldably formed to extend to one end of the main body unit, a monitor unit mounted on the monitor mount, a heating unit located adjacently to the endoscope unit, and a data transmitting and receiving unit installed in the endoscope unit.

### CROSS-REFERENCE TO RELATED APPLICATION

This application claims the priority benefit of Korean Patent Application No. 10-2023-0113504, filed on Aug. 29, 2023, which is hereby incorporated by reference in its entirety.

### BACKGROUND OF THE INVENTION

The present disclosure relates to a dental endoscope system.

In general, the endoscope system used in the otolaryngology area is built into a unit chair, and unlike the otolaryngology, which uses only one unit chair, in the case of dentistry, which uses multiple unit chairs, a movable type endoscope system is essential. Since such a movable type endoscope system can be used smoothly only when the power cable, video cable for viewing real-time images, and information transmission cable for accessing stored images are always connected, it should be used by connecting the three cables each time and then turning the power on. However, since the booting and preparation time takes 5 minutes or more in this case, the time required to prepare each time for 1 to 2 minutes of use is relatively long, and when cable A1 are positioned on the floor as shown in FIG. 1, there have been problems in that the movement of a chair or an appliance was not only restricted, but also accidents causing patients to trip and fall occur.

Therefore, there is an urgent need for a method capable of wirelessizing the three cables mentioned above.

### PRIOR ART DOCUMENT

### Patent Document

(Patent Document 1) Korean Patent No. 10-2093426 (2020.03.19)

### SUMMARY OF THE INVENTION

### Technical Problem

An object of the present disclosure, which has been derived to solve the problems described above, is to provide a dental endoscope system that is provided with a power supply unit for its own power supply along with an endoscope unit, thereby enabling three cables to be made wireless.

### Technical Solution

In order to achieve the above object, a dental endoscope system according to an embodiment of the present disclosure includes a movable main body unit, a power supply unit provided in the main body unit, an endoscope unit connected to the power supply unit, a monitor mount which is rotatably and foldably formed to extend to one end of the main body unit, a monitor unit mounted on the monitor mount, a heating unit located adjacently to the endoscope unit, and a data transmitting and receiving unit installed in the endoscope unit.

### Advantageous Effects

As described above, the dental endoscope system according to the embodiment of the present disclosure is provided with a power supply unit for its own power supply along with the endoscope unit, thereby wirelessizing the dental endoscope system so it is not only easy to move, but also can improve patient safety and reduce booting and preparation time.

Further, it can secure a clear image while providing sterilization to the endoscope unit by positioning the heating unit adjacently to the endoscope unit.

### BRIEF DESCRIPTION OF DRAWINGS

FIG. 1 is a photograph showing the state of use of a conventional dental endoscope.

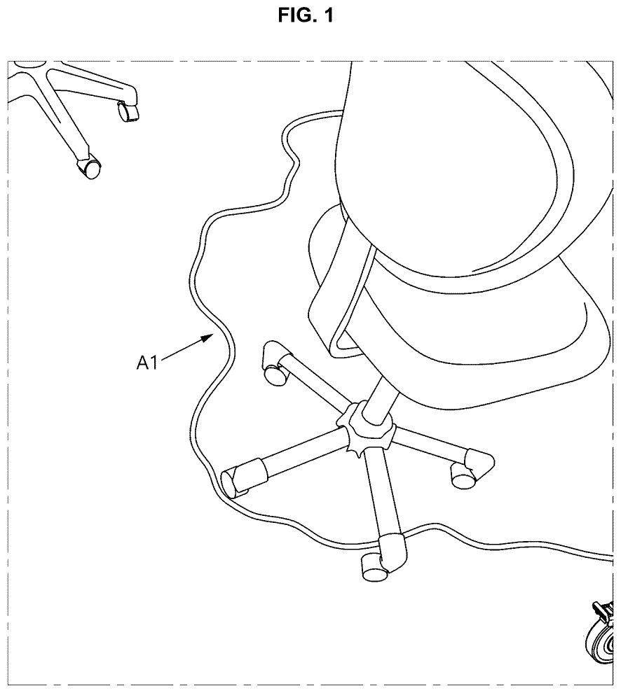

FIG. 2 is a drawing schematically showing the dental endoscope system of the present disclosure, and FIG. 3 is a photograph showing one side of the dental endoscope system of the present disclosure.

FIG. 4 is photographs before and after coupling an endoscope camera and a sinuscope to a cable and an optical cable, respectively.

FIG. 5 is a photograph of the monitor composition seen from the operator's position.

FIG. 6 is a composition photograph showing the operator's field of view and the patient's field of view.

### DETAILED DESCRIPTION OF THE INVENTION

Hereinafter, embodiments of the present disclosure are described in detail. However, the present disclosure is not limited to the embodiments disclosed below, but may be implemented in various different forms. The present embodiments are only provided to ensure that the disclosure of the present disclosure is complete and to more completely inform those skilled in the art of the content of the present disclosure.

Hereinafter, the dental endoscope system of the present disclosure will be described in detail.

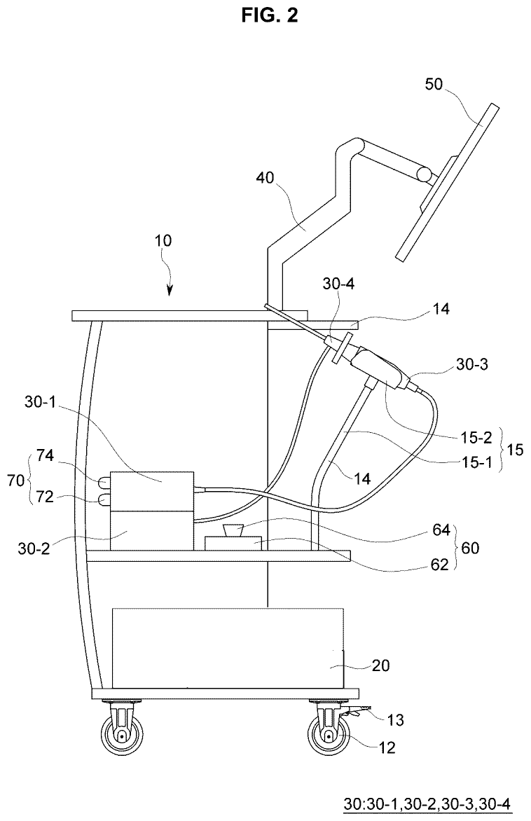

Referring to FIG. 2, the dental endoscope system 1 according to an embodiment of the present disclosure includes a main body unit 10, a power supply unit 20, an endoscope unit 30, a monitor mount 40, and a monitor unit 50, a heating unit 60, and a data transmitting and receiving unit 70.

The main body unit 10 may be provided with one or more driving wheels 12 at the bottom so that it can move freely, and may be provided with, for example, a pair of front wheels and a pair of rear wheels. Meanwhile, a locking member 13 is installed on one side of the driving wheel 12, and is locked when in use to prevent the driving wheel 12 from rolling and moving, and when movement is desired, locking is released so that the driving wheel 12 is rolled and can move to the desired location while freely rotating. One side of the main body unit 10 may be provided with one or more moving handles 14 used when moving as shown in FIG. 3 for stable movement.

The main body unit 10 may include a holder 15 on which the endoscope unit 30, which will be described later, is mounted, and the holder 15 may include a support member which has its one end installed on the main body unit 10 and has one or more bending areas and a coupling member 15-2 which is connected to the other end of the support member 15-1 and interference fitting-coupled to the endoscope unit 30. The coupling member 15-2 may be made of an elastic yet sturdy material for easier coupling with the endoscope unit 30, and may be made of one or more selected from polyurethane, polyester, and epoxy in this embodiment. In addition, a plurality of protrusions may be provided on the coupling member 15-2 to further increase the coupling strength by widening the surface area with the endoscope unit 30, and at this time, the plurality of protrusions may be formed of rubber, silicone, and a mixture thereof, but may be formed as one piece, thereby increasing robustness.

The power supply unit 20 is provided in the main body unit 10 and may include a battery pack. The power supply unit 20 supplies power to the endoscope unit 30, monitor unit 50, heating unit 60, and data transmitting and receiving unit 70, and in order to ensure movement safety according to the center of gravity of the main body unit 10, it is preferable to dispose it in a diagonal direction with the monitor mount 40 and the monitor unit 50, which will be described later. Such a power supply unit 20 can solve problems caused by external connection wires, which not only makes it easier to move the main body unit 10, but also improves patient safety and reduces booting and preparation time. For example, the power supply unit 20 may further include a converter to enable charging of all products in addition to the battery pack.

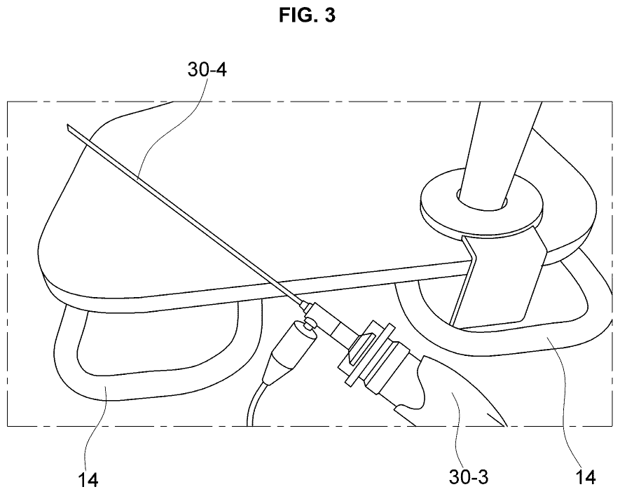

The endoscope unit 30 may include an endoscope set-top 30-1, a light source 30-2, an endoscope camera 30-3, and a sinuscope 30-4. The light source 30-2 may be located on the bottom side of the endoscope set-top 30-1, and the endoscope set-top 30-1 and the light source 30-2 may be connected to the power supply unit 20 to receive power. The endoscope camera 30-3 may be connected to the endoscope set-top 30-1 in the rear direction through a cable, and the sinuscope 30-4 may be formed to be connected to one side of the endoscope camera 30-3, and may be connected to the light source 30-2 through an optical cable, specifically connected vertically and used. As shown in FIG. 4, the endoscope camera 30-3 and the sinuscope 30-4 are used as a combined body that is a state in which they are connected to a cable 80 and an optical cable 90, respectively, and it may usually be used by holding the endoscope camera 30-3 by hand. When using the endoscope camera 30-3, it can be used separately from the holder 15, and after use is completed, it can be stored by mounting it on the holder 15. Since fogging may occur to decrease the clarity of the image if the sinuscope 30-4 is entered directly into the oral cavity because the inside of the oral cavity has high temperatures and high humidities, the sinuscope 30-4 should be maintained at a temperature higher than the patient's body before the procedure. It took a relatively long time as the temperature of the sinuscope 30-4 was raised with a heater in the past, but such a problem was solved by applying a method of immersing the sinus endoscope 30-4 in a sterilizing solution in this embodiment, and the heating unit 60 will be described below.

The monitor mount 40 may be rotatably and foldably coupled to one end of the main body unit 10 in order to adjust the position and direction of the monitor unit 50, and for example, it may be provided at the right front corner of the main body unit 10 in consideration of convenience of use. In this embodiment, in order to ensure the safety of the center of gravity of the main body unit 10, it is preferable that the monitor mount 40 be installed in a different direction from the power supply unit 20.

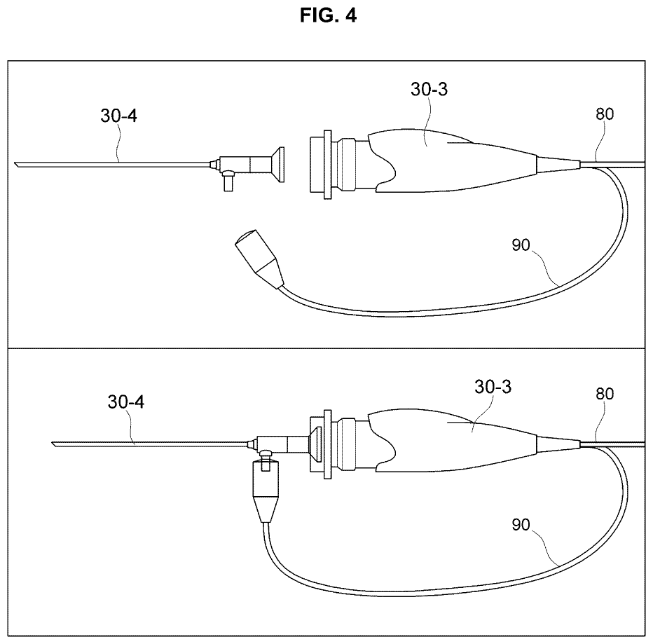

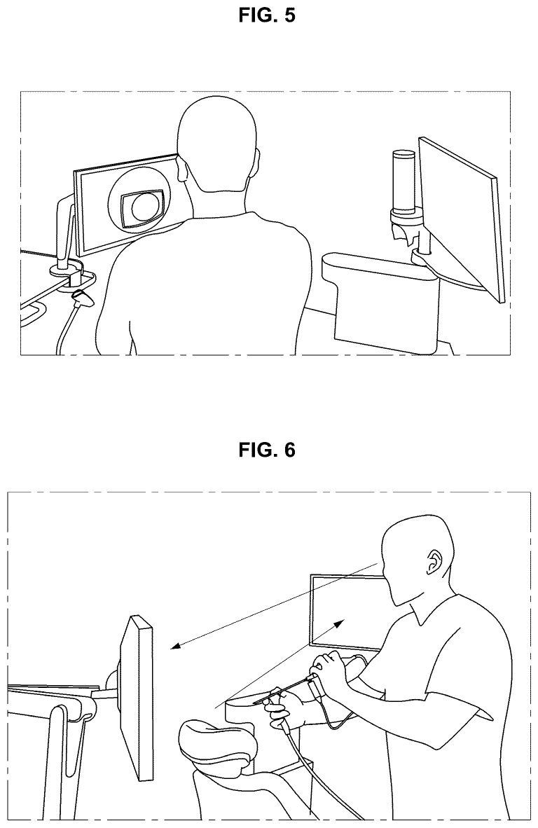

The monitor unit 50 may be mounted on the monitor mount 40 to help the operator proceed the task by reproducing the image received from the endoscope unit 30, and the location of the monitor unit 50 may be adjusted through the monitor mount 40. Referring to FIGS. 5 and 6, since the operator is usually located on the left side of the patient and performs the procedure while looking to the right, it may be advantageous that the monitor unit 50 is located on the right side of the operator in terms of being able to secure space for the assist as much as possible while securing the operator's natural gaze direction.

In order to receive power from the power supply unit 20 and facilitate use of the endoscope camera 30-3, the heating unit 60 may be located adjacently to the endoscope unit 30 and may include a heat transfer plate 62 and a container 64. The container 64 is provided on the top of the heat transfer plate 62, and contains a sterilizing solution, thereby maintaining the temperature, and on the other hand, the sinuscope 30-4 may be immersed and sterilized before and after the procedure. Such a sterilizing solution is heated to 60 to 80° C. by the heat transfer plate 62 and provided so that when the sinuscope 30-4 is immersed in it, the sinuscope 30-4 may be heated to a temperature higher than the body within a short period of 2 to 3 seconds to enable the efficiency of the procedure to be increased, and fogging of the sinuscope 30-4 may be prevented during the procedure to enable clear image reading. For example, the container 64 may be made of iron-nickel-chromium based austenitic stainless steel.

The data transmitting and receiving unit 70 may include an image transmitter and receiver 72 and a file transmitter and receiver 74 which are installed in the endoscope unit 30, and specifically, are installed on one side of the endoscope set-top 30-1, and collect, store, and transmit and receive digital images and files. Accordingly, the image is transmitted in real time, and as shown in FIG. 6, the image is received in real time on a monitor visible to the patient's field of vision, allowing the patient to understand his or her disease in detail while viewing the image. In some cases, the operator may also proceed with the procedure while looking at the monitor visible to the patient's field of vision. For example, the file transmitter and receiver 74 wirelessly shares files stored in the endoscope set-top 30-1 so that they can be checked on a desktop or Pad. This may not only facilitate the creation of medical records but also allow comparison of past images and current conditions to proceed conveniently. The configuration of the data transmitting and receiving unit 70 as described above makes it easy to share information so that it may not only improve the medical environment but also improve the quality of medical services.

Hereinabove, exemplary embodiments of the present disclosure have been described in detail above, but the scope of rights of the present disclosure is not limited thereto, and various modifications and improved forms made by those skilled in the art using the basic concept of the present disclosure defined in the following claims also fall within the scope of rights of the present disclosure.

All technical terms used in the present disclosure, unless otherwise defined, are used with the same meaning as commonly understood by a person skilled in the art in the field related to the present disclosure. The contents of all publications described as reference documents in this specification are introduced into the present disclosure.

### EXPLANATION OF REFERENCE NUMERALS

1: Dental endoscope system
10: Main body unit, 12: Driving wheel
13: Locking member, 14: Moving handle
15: Holder, 15-1: Support member
15-2: Coupling member, 20: Power supply unit
30: Endoscope unit, 30-1: Endoscope set-top
30-2: Light source, 30-3: Endoscope camera
30-4: Sinuscope, 40: Monitor mount
50: Monitor unit, 60: Heating unit
62: Heat transfer plate, 64: Container
70: Data transmitting and receiving unit, 72: Image transmitter and receiver
74: File transmitter and receiver
80: Cable
90: Optical cable

1. A dental endoscope system comprising:
a movable main body unit;
a power supply unit provided in the main body unit;
an endoscope unit connected to the power supply unit;
a monitor mount which is rotatably and foldably coupled to one end of the main body unit;
a monitor unit mounted on the monitor mount;
a heating unit located adjacently to the endoscope unit; and
a data transmitting and receiving unit installed in the endoscope unit.

2. The dental endoscope system of claim 1, wherein the main body unit comprises a holder on which the endoscope unit is mounted, and the holder comprises a support member which has its one end installed on the main body unit and has one or more bending areas and a coupling member which is connected to the other end of the support member and interference fitting-coupled to the endoscope unit.

3. The dental endoscope system of claim 1, wherein the endoscope unit comprises:
an endoscope set-top provided in the main body unit;
a light source located on the bottom side of the endoscope set-top;
an endoscope camera connected to the endoscope set-top; and
a sinuscope formed to be connected to one side of the endoscope camera.

4. The dental endoscope system of claim 1, wherein the heating unit comprises:
a heat transfer plate; and
a container which is provided on the top of the heat transfer plate, and contains a sterilizing solution.

5. The dental endoscope system of claim 1, wherein the data transmitting and receiving unit comprises an image transmitter and receiver and a file transmitter and receiver.
